(19) United States
(12) Patent Application Publication
(10) Pub. No.: US 2019/0046034 A1
(43) Pub. Date: Feb. 14, 2019

(54) SYSTEMS AND METHODS FOR RETRANSMITTING WIRELESS DATA STREAMS

(71) Applicant: KONINKLIJKE PHILIPS N.V., EINDHOVEN (NL)

(72) Inventors: Calvert Tazewell HAWKES, SARASOTA, FL (US); Michael Angelo GEMMATI JR, OVIEDO, FL (US); Carolus Gerardus THIJSSEN, MAITLAND, FL (US)

(21) Appl. No.: 16/078,030

(22) PCT Filed: Feb. 23, 2017
(86) PCT No.: PCT/IB2017/051023

§ 371 Date: Aug. 21, 2018

Related U.S. Application Data
(60) Provisional application No. 62/298,641, filed on Feb. 23, 2016.

(51) Int. Cl. A61B 5/00; H04L 1/18

(52) U.S. Cl. CPC A61B 5/0017; H04L 1/1812; A61B 5/0402; A61B 5/002; A61B 5/0046; A61B 5/0026

(57) ABSTRACT

An apparatus (10) for processing physiological data streams broadcast by at least one associated medical information sensor (18) is provided. The apparatus includes: a first hub (6) including a first radio (12) configured to receive an individual medical information data stream broadcast by the at least one associated sensor (18) at a data stream carrier frequency and to transmit a non-electrical signal carrying the individual medical information data stream; a second hub (8) including a second radio (14); and a non-electrical communication link (16) between the first radio (12) and the second radio (14) via which the non-electrical signal carrying the individual medical information data stream is communicated from the first radio (12) to the second radio (14). The second radio (14) is configured to receive the non-electrical signal carrying the medical information data stream from the first radio (12) and re-broadcast the medical information data stream at the carrier data stream frequency with a defined time lag respective to the broad cast by the at least one associated sensor (18)

### FIELD

The following relates to the medical imaging arts, magnetic resonance imaging arts, data transmission arts, and related arts.

### BACKGROUND

Wireless data is transmitted over radio frequencies in a hospital environment by sensors, providing remote monitoring of physiological parameters such as blood oxygen saturation (SpO2), electrocardiograms, temperature, blood pressure, and the like. For example, such wireless vital sign sensors are increasingly being used to monitor patients undergoing magnetic resonance imaging (MRI) procedures. Wireless sensors have a particular advantage in the MRI setting since a wired sensor connected with the patient in the MRI bore can pick up time-varying magnetic fields from radio frequency (RF) transmissions and/or magnetic field gradients applied during the MRI imaging. Such signal pickups can introduce noise and in severe instances can lead to heating and potential consequent patient injury.

During an MRI scan the sensors are connected to the patient. A monitor located in the magnet room can display the patient information. Some typical implementations employ a 2.4 GHz radio with relatively short range (i.e., 50-100 feet) to transmit vital sign data off the sensor unit. The wireless sensor transmissions are received by the radio of a patient monitor located inside the magnet room and displayed. However, in practice the radiological technician or other medical professional conducting the MRI examination may not be present in the magnet room during the imaging, but rather may be located in an adjacent MRI control room. The MRI machine is controlled from the control room, so the monitor is the control room for convenience. In addition, noise produced from an MRI machine during scanning can be noisy, so the technician prefers to keep the monitor in the quieter control room. It would be useful to be able to wheel the patient monitor from the magnet room into the control room to enable the technician to continue to view patient vital signs in real time, in the control room, during the MRI procedure. However, the magnet room is enclosed in a room-sized Faraday shield to limit detrimental emissions from the MRI apparatus and to limit outside RF interference from adversely impacting the MRI imaging. RF shielding of the magnet room interferes with, or entirely prevents, the wireless sensor transmissions from being received by the patient monitor in the control room.

A related problem is that it may be desirable to send patient data to a Hospital Information System (HIS). The shielding of the magnet room makes this difficult.

The following provides new and improved apparatuses and methods which overcome the foregoing problems and others.

### BRIEF SUMMARY

In accordance with one aspect, an apparatus for processing physiological data streams broadcast by at least one associated physiological sensor is provided. The apparatus includes: a first hub including a first radio configured to receive an individual medical information data stream broadcast by the at least one associated sensor at a data stream carrier frequency and to transmit a non-electrical signal carrying the individual medical information data stream; a second hub including a second radio; and a non-electrical communication link between the first hub and the second hub via which the non-electrical signal carrying the individual medical information data stream is communicated from the first radio to the second radio. The second radio is configured to receive the non-electrical signal carrying the medical information data stream from the first radio and re-broadcast the medical information data stream at the data stream carrier frequency with a defined time lag respective to the broadcast by the at least one associated sensor.

In accordance with another aspect, a method for processing physiological data streams broadcast by at least one associated physiological sensor is provided. The method includes: receiving, with a first radio in the first hub, an individual physiological data stream broadcast by the at least one associated physiological sensor at a data stream carrier frequency; transmitting, with the first radio, a non-electrical signal carrying the individual physiological data stream to a second radio via a non-electrical communication link between the first radio and the second radio; receiving, with the second hub, the non-electrical signal carrying the physiological data stream from the first radio; and re-broadcasting, with the second radio, the physiological data stream at the data stream carrier frequency with a defined time lag respective to the broadcast by the at least one associated physiological sensor.

In accordance with another aspect, an apparatus for retransmitting data streams is provided. The apparatus includes at least one first physical electrical data port configured to receive an individual patient data stream output at a physical electrical data port of at least one associated medical device and to transmit a non-electrical signal carrying the individual patient data stream. At least one second physical electrical data port physically mimics the physical electrical data port of the at least one associated medical device. A fiber optic cable connects the first and second physical electrical data ports together to carry the non-electrical signal from the at least one first physical electrical data port to the at least one second physical electrical data port. The at least one second physical electrical data port is further configured to receive the non-electrical signal carrying the patient data stream from the at least one first physical electrical data port and output the patient data stream whereby the physical electrical data port of the at least one associated medical device is mimicked at the at least one second physical electrical data port.

One advantage resides in uninhibited transmission of a broadcasted signal and a re-broadcasted signal between multiple rooms.

Another advantage resides in moving a patient monitor between multiple rooms without interrupting transmission of data to the patient monitor.

Another advantage resides in combining multiple data streams into a single data stream and transmitting the single data stream between multiple rooms.

Another advantage resides in using a fiber optic cable to transmit data between multiple rooms.

Further advantages of the present invention will be appreciated to those of ordinary skill in the art upon reading and understand the following detailed description. It will be appreciated that any given embodiment may achieve none, one, more, or all of the foregoing advantages and/or may achieve other advantages.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 shows an embodiment of an apparatus to retransmit data streams in accordance with one aspect of the present disclosure.

FIG. 2 shows an example of an operation of the apparatus of FIG. 1.

FIG. 3 shows a flowchart showing an exemplary method of use of the apparatus of FIG. 1.

### DETAILED DESCRIPTION

Embodiments disclosed herein extend the wireless sensor communication range outside of the Faraday cage-shielded magnet room to neighboring areas (e.g. the MRI control room) while minimizing modifications to the patient monitor and maintaining integrity of the RF shielding of the magnet room. To this end, two radios can be provided—one inside the magnet room and one outside the magnet room (e.g. inside the adjacent control room)—which are connected by an optical fiber link passing through the RF shielding. The inside (or “first”) radio picks up sensor transmissions and communicates them to the outside (or “second”) radio via the fiber optic link, and the outside radio re-broadcasts the same transmissions at their original carrier frequencies but with a predefined time lag.

With this approach, only software modifications are needed at the patient monitor, e.g. adjusting the firmware to check the outside radio repeater time slots if the primary sensor signal becomes unreliable. The re-broadcasts are preferably at the same carrier frequency and use the same modulation and data encoding so no modification to the radio hardware is needed. The predefined time lag ensures that if both the broadcast and the re-broadcast are in range (e.g., as may be the case if the door between the control room and the magnet room is open) then the original sensor broadcast and the rebroadcast can be distinguished. Various approaches can be used to select the signal (broadcast or rebroadcast) for use at the patient monitor. In one approach, the broadcast is used unless its signal strength is too low (or the signal is poor by some other metric, e.g. fraction of bad packets) at which point the monitor switches to the rebroadcast. In another approach, the strongest signal is chosen.

To handle multiple data sources simultaneously, whether from wireless sensors or medical devices attached to physical ports, a multiplexor is provided with the inside radio to multiplex the sensor signals onto the optical fiber link. Each sensor usually broadcasts on a different channel (i.e. different carrier frequency), and the same solution for signal differentiation currently used by the patient monitor to capture these sensor signals at different frequencies can be replicated at the inside radio. One suitable approach includes leveraging the ECG signal having large redundancies (transmits 3000 points/sec, ECG trace can be reconstructed with 200 points/sec) and the use of two antennae to employ antenna diversity to compensate RF reflections in the magnet room. Another approach, albeit costlier in hardware, is to provide a set of receivers at the patient monitor for the different sensor channels. At the outside radio, the multiplexed signal is demultiplexed and a similar approach is used to rebroadcast the original sensor signals at the appropriate radio frequencies. It will also be appreciated that analogous hardware could be used to rebroadcast a signal originating in the control room in the magnet room.

In another embodiment, the outside radio is replaced by or augmented with one or more physical electrical data ports (Ethernet, serial, etc.) that mimic output physical electrical data ports of medical devices located inside the magnet room. The inside radio is similarly replaced by or augmented with mating electrical data ports designed to connect with the physical electrical data ports of the medical devices. In this way, the medical devices can be plugged into the mating ports located inside the magnet room, the signals from the ports are bundled together and transmitted over the non-electrical fiber optic link, and are de-bundled outside the magnet room and the constituent signals distributed to the appropriate physical electrical data ports mimicking the instrument electrical data ports. Thus, a user can “plug into” the medical device remotely, e.g. in the control room, by plugging into the output physical electrical data port of the outside radio corresponding to the physical port of the medical device located inside the magnet room. The addition of a Hospital Information System (HIS) port at the outside is also contemplated, so that signals from instruments inside the magnet room can be recorded in the HIS. Note that in this embodiment there is no need for the predefined time lag.

The use of a fiber optic link is advantageous for connecting the inside and outside radios due to its high speed and bandwidth (e.g. 5 megabits/sec readily achievable) which facilitates maintaining the design-basis time lag between the original sensor broadcast and the re-broadcast, although other RF shielding-compliant non-electrical links such as a laser/window/photodiode optical link are also contemplated.

One embodiment includes a digital repeater comprising an RF transceiver located in the magnet room, a fiber optic link through a waveguide to the control room, and an RF transceiver located in the control room. Data transmission in the control room is time synchronized so as to allow for a receiving device such as a monitor to seamlessly receive data in the magnet room, control room, or somewhere in between. In addition, a data port is provided in the control room radio to allow the received data to be sent to a HIS.

As used herein, the term “broadcast” (and variants thereof) refers to as a non-directional wireless transmission (as opposed to a point-to-point directed wireless transmission, e.g. using a directional beam antenna or a laser beam) that can be received by any suitably tuned radio within a broadcast range of the transmitter. For example, the term “broadcast” can refer to a transmission of a signal from a physiological sensor to a first radio.

As used herein, the term “re-broadcast” (and variants thereof) refers to as a non-directional wireless transmission that can be received by any suitably tuned radio within a predefined (re-)broadcast range. For example, the term “re-broadcast” can refer to a transmission of a signal from a second radio to a patient monitor.

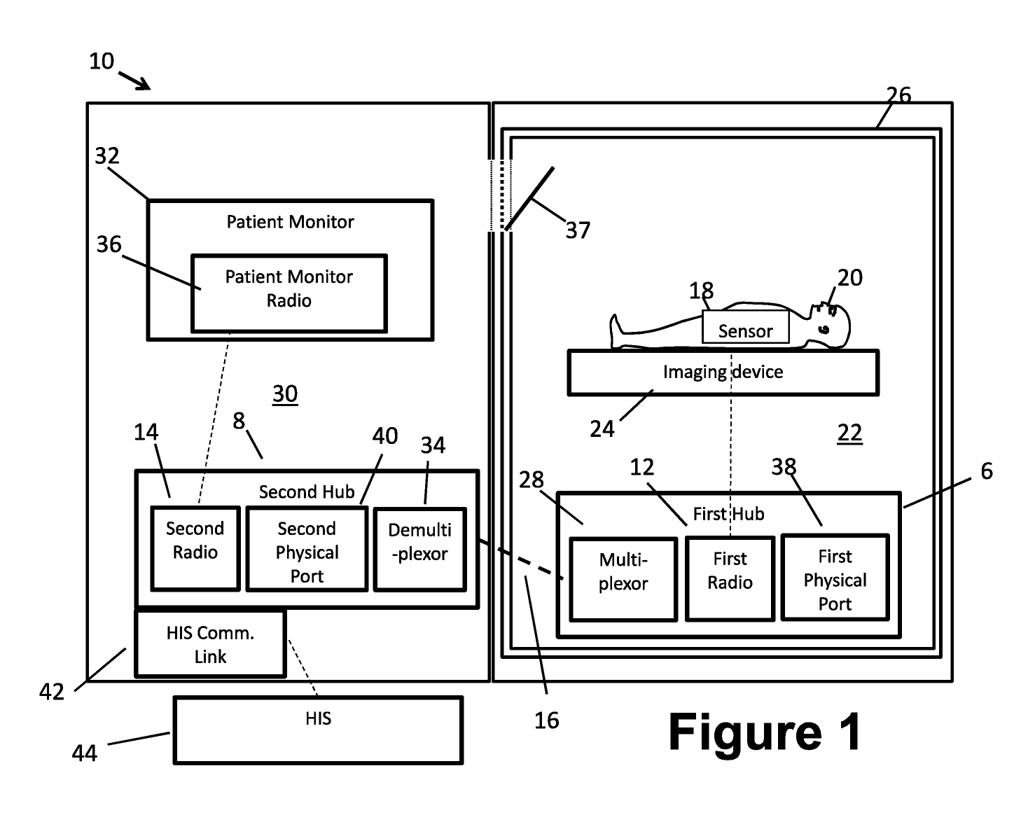

With reference to FIG. 1, an embodiment of an apparatus 10 for processing physiological data streams is shown. As shown in FIG. 1, the apparatus 10 includes a first hub 6 with a first radio 12, a second hub 8 with a second radio 14, and a non-electrical communication link 16 between the first and second hubs. Each of these components is described in more detail below.

The first radio 12 is configured to receive an individual medical information data stream broadcast by the at least one associated physiological sensor 18 at a data stream frequency and to transmit a non-electrical signal carrying the individual physiological data stream. For example, the medical information can include information about the patient, including physiological data obtained by the sensor 18, information related to battery life, software version, drub library version, malfunction alerts, general alerts, high bandwidth data, and the like. A patient 20 can be positioned in a first room 22 with an imaging device 24 (e.g., a magnetic resonance (MR) imaging device, a positron emission tomography (PET) imaging device, a single-positron emission computed tomography imaging device, and the like). It should be noted that the imaging device 24 is diagrammatically indicated in FIG. 1, and may for example be a horizontal bore-type MRI with the patient 20 loaded into the MRI bore (and hence substantially occluded from view), an open MRI, or so forth. The first room 22 includes a radio frequency (RF) shield 26 that surrounds the first room (e.g., by being embedded in walls of the room). Such a radio frequency shield is commonly used in conjunction with an MRI and, as such, the first room 22 can be referred to as a “magnet room” and the RF shield 26 forms a Faraday cage 26 enclosing the magnet room 22. However, other types of imaging systems may benefit from Faraday cage shielding, e.g. a PET scanner may be sensitive to outside RF interference. The physiological sensors 18 (e.g., an SpO2 sensor, an ECG sensor, a temperature sensor, a blood pressure sensor, and the like) can be operably connected to the patient 20. In the case of sensors 18 used to monitor a patient 20 loaded into the MRI examination region and hence exposed to strong magnetic fields, magnetic field gradients, and RF pulses, the sensor should be MRI compatible. Using a wireless sensor is one way to improve MRI compatibility as this eliminates electrically conductive wires that could otherwise couple with magnetic field gradients and heat up due to induced eddy currents. The sensor 18 for MRI compatibility is also preferably free of ferromagnetic material and may incorporate other features such as perforated RF shielding (if such shielding is needed) to suppress eddy currents.

The first radio 12 is configured to receive physiological data streams from each of the physiological sensors 18 and transmit the data streams to the second radio 14. To do so, the first hub 6 includes a multiplexor or bundler 28. The multiplexor 28 is programmed to receive a plurality of individual physiological data streams from the physiological sensors 18. For example, the multiplexor 28 is programmed to receive an SpO2 signal from the SpO2 sensor, an ECG signal from the ECG sensor, and so on. The multiplexor 28 is then programmed to bundle each of these physiological data streams into a single data stream. For example, the multiplexor 28 bundles the individual data streams into a non-electrical signal. Once the single data stream is generated, the multiplexor 28 is then programmed to transmit the non-electrical signal carrying the single data stream via the non-electrical communication link 16 to the second radio 14.

The second hub 8 is configured to receive the non-electrical signal carrying the physiological data stream from the first radio 12 and re-broadcast using the second radio 14 the physiological data stream at the data stream frequency with a defined time lag respective to the broadcast by the physiological sensors 18. The second radio 14 is located in a second room 30 that is separate from the first room 22. The second room 30 does not include an RF shield. As such, the second room 30 can be referred to as a “control room.”

The second radio 14 is configured to receive the single, bundled data stream from the first radio 12, and transmit the data stream to a patient monitor 32 (described in more detail below). To do so, the second hub 8 includes a de-multiplexer or de-bundler 34. The de-multiplexor is programmed to receive the bundled, single data stream from the non-electrical communication link 16 via the multiplexor 28 of the first radio 12. For example, the de-multiplexor 34 is programmed to de-bundle the single data stream into the individual data streams. For example, the de-multiplexor 34 de-bundles the single data stream back into the individual data streams originally transmitted by the physiological sensors 18 to the first radio 12. Once the single data stream is de-bundled, the de-multiplexor 34 is then programmed to retransmit the individual data streams to the patient monitor 32, as described in more detail below.

The non-electrical communication link 16 is configured to connect the first hub 6 and the second hub 8. For example, the non-electrical communication link 16 is configured to be operably connected to the first radio 12 in the magnet room 22, and the first second radio in the control room 30 by passing through the RF shield 26 (i.e., through a wall separating the magnet and control rooms 22 and 30). As a result, the non-electrical signal carrying the individual physiological data stream is communicated from the first radio 12 to the second radio 14. In some embodiments, the non-electrical communication link 16 can be configured as a fiber optic cable. Advantageously, the use of a fiber optic link is preferred due to its high speed and bandwidth (e.g. 5 megabits/sec readily achievable) which facilitates maintaining the design-basis time lag between an original sensor and a re-broadcast, as described in more detail below.

It should be noted that the inside and outside radios 12, 14 are shown diagrammatically in FIG. 1. In a preferred implementation, each radio is a compact transceiver small enough to fit into a small space, e.g. the size of a cellular telephone (cellphone) in some embodiments, disposed in a corner of the respective magnet room 22 or control room 30 or mounted on a wall thereof, so as to be out-of-the-way of the radiological technician or other personnel. The two radios 12, 14 are preferably (although not necessarily) placed close to each other on opposite sides of the wall between the adjoining magnet room 22 and control room 30, so that the optical fiber 16 can be of short length.

In some embodiments, the apparatus 10 can include a patient monitor 32. The patient monitor 32 is configured to receive the individual physiological data streams broadcast by the physiological sensors 18 at the data stream frequency at which the sensors transmit the data streams to the first radio 12. To do so, the patient monitor 32 includes a patient monitor radio 36 configured to receive the data streams from the sensors 18. In addition, the patient monitor radio 36 is configured to receive a re-broadcast from the second radio 14 of the physiological data stream at the data stream carrier frequency with the defined time lag. The patient monitor 32 is typically positioned in the magnet room 22 so that medical professionals in the control room 30 can view the sensor data on the patient monitor. Advantageously, the patient monitor 32 can be configured to be mobile (e.g. battery-powered and mounted on a wheeled support rack or wheeled support console) so that it can be moved back and forth from the magnet room 22 and the control room 30 via a door 37 (again it is to be understood that the FIG. 1 is diagrammatic, and the patient monitor 32 and door 37 are respectively sized to allow the patient monitor to be wheeled through the door), thereby ensuring that the data streams from the sensors 18 are continuously transmitted and shown on the patient monitor. In another example, the patient monitor 32 can be configured as a hand-held device, thereby allowing a technician to walk back and forth between the magnet room 22 and the control room 30 while carrying or holding the patient monitor.

Stated another way, the patient monitor 32 is configured to receive: (1) an original broadcast of the data streams from the sensors 18; and (2) a re-broadcast of the same data streams from the second radio 14. The patient monitor 32 is configured to select one of the broadcast and the re-broadcast and to read the physiological data stream from the selected one of the broadcast and the re-broadcast. In one example, the patient monitor 32 is configured to select one of the broadcast and the re-broadcast based on a signal strength metric of at least the broadcast. In one such approach, the broadcast is used unless its signal strength is too low (or the signal is poor by some other metric, e.g. fraction of bad packets) at which point the monitor switches to the rebroadcast. In another signal strength-based approach, the strongest signal is chosen. It will be appreciated that because of the Faradays shield 26, the strongest signal is likely to be the direct broadcast from the sensor 18 when the patient monitor 32 is located inside the magnet room 22; whereas, the strongest signal is likely to be the re-broadcast from the second radio 14 when the patient monitor 32 is located in the control room 30. If the patient monitor 32 is located in the doorway of the door 37 then both the broadcast and the re-broadcast may be received with relatively strong signals, and one signal is selected preferably on the basis of signal strength. It will be noted that even if the door 37 is merely open this may be enough to reduce effectiveness of the Faraday cage 26 to an extent allowing both the broadcast and re-broadcast to be received at the patient monitor 32.

Although signal strength is generally a preferred basis for selecting between the broadcast and re-broadcast, other criteria are contemplated to be used in the selection. For example, if the broadcast is initially selected and processed but a checksum data verification fails, then the re-broadcast may instead be processed. In some embodiments, as shown in FIG. 1, the first hub 6 includes at least one first physical electrical data port 38 corresponding to one or more associated medical devices (e.g., an infusion pump, an anesthesiology machine, and the like). For example, the first physical electrical data ports 38 typically contain patient data for the associated medical device (e.g., data for an infusion pump that can include the type of drug, concentration, time start, alarm, etc.). Each first electrical data port 38 is configured to receive physiological data from a medical device in the magnet room 22 (not shown, e.g., an infusion pump, anesthesiology machine, or so forth). Similarly, the second hub 8 includes at least one second physical electrical data port 40 corresponding to each of the first ports 38, and, as a result, are associated with the medical devices (such as the infusion pump, the anesthesiology machine, and the like). Each second physical electrical data port 40 is configured to receive the physiological data from the corresponding first physical electrical data port 38 via the non-electrical communication link 16. Each second physical electrical data port 40 is designed to mimic the output port of a medical device for which it serves as a “remote surrogate” output. Thus, for example, the second physical electrical data port 40 has the same connector type and outputs data using the same format as the output port of the medical device. In this way, a user can plug into the second physical electrical data port 40 in the control room 30 and receive data identically to the way the user would plug into the output port of the medical device located inside the magnet room 22.

In other embodiments, referring back to FIG. 1, the apparatus 10 can also include a hospital information system communication link 42 configured to transmit data from the second hub 8 to an associated hospital information system 44. For example, the data streams sent by the sensors 18 can be transmitted to the hospital information system communication link 42 so that the hospital information system 44 can be advantageously continuously updated with information related to the patient 20. In other words, the data sent to the hospital information system 44 may include a patient identifier and the time. In another example, when a patient identification is not known, a machine ID for the sensors 18 or medical devices (not shown) can be used. The hospital information system 44 can resolve the patient identification since the hospital information system knows which patient was connected to which sensor 18 or medical device at a selected time. The hospital information system communication link 42 can be configured as a serial port. In some embodiments, the hospital information system communication link 42 is disposed on a portion of the second radio 14; however, it will be appreciated that the hospital information system communication link 42 can be disposed in any suitable location (e.g., on the patient monitor 32, separately from the components of the apparatus 10, and the like).

It will also be appreciated that the various signals and values describe herein can be communicated to the various components 12, 14, 18, 32 and data processing components 28, 34 via a communication network (e.g., a wireless network, a local area network, a wide area network, a personal area network, BLUETOOTH®, and the like). In another contemplated embodiment, the output of the sensors 18 is suitably displayed on the patient monitor 32.

### EXAMPLE

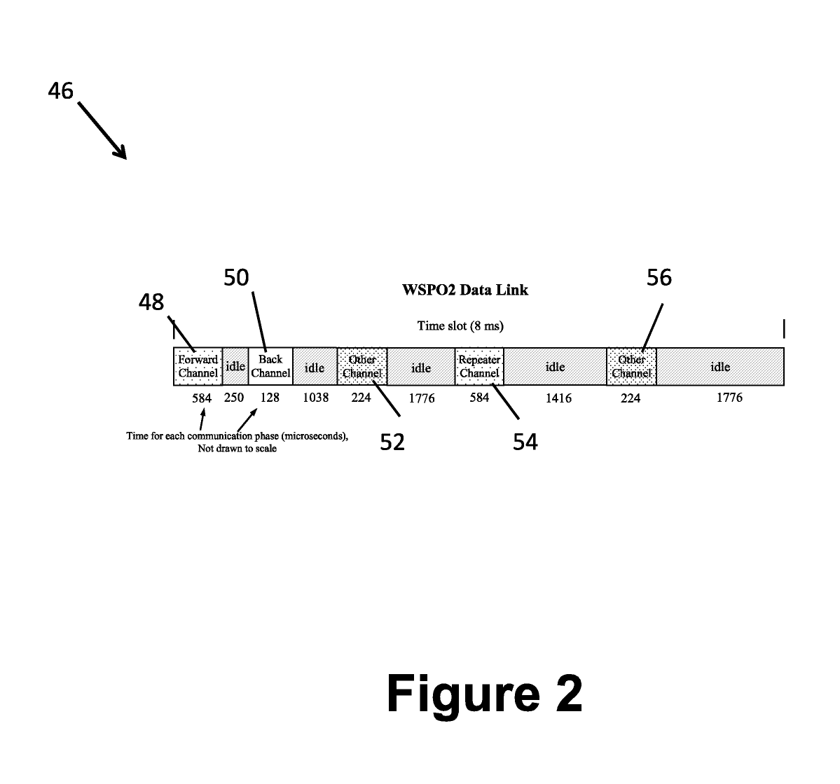

FIG. 2 shows an example of a time line 46 during the operation of the apparatus 10. In this example, the only physiological sensor 18 connected to the patient 20 is an SpO2 sensor. The sensor 18 is configured to transmit a plurality of data packets in a forward channel at 125 Hz. At this frequency, one packet is transmitted every 8 milliseconds (i.e., 8000 microseconds, which is the length of the time line 46) on a defined carrier frequency F_{SPO2}. (It is to be understood that the carrier frequency is different from the packet frequency. For example, if a 2.4 GHz radio is being used then the carrier frequency is around 2.4 GHz; whereas, the packet frequency in this example is 125 Hz.) The sensor 18 is configured to transmit the data packets to the first radio 12 in the forward channel during a forward channel time period 48, which lasts approximately 584 microseconds, and contains a rolling window of three successive samples. After an idle time of approximately 250 microseconds, the sensor 18 receives data in a back channel during a back channel time period 50, which lasts approximately 128 microseconds long. The speed of the fiber data transmission is chosen to be at a sufficient rate to allow time synchronization between packet protocol at the first radio 12 and packet protocol at the second radio 14; such as 5 Mbps high speed serial or 100 Mbps Ethernet packet protocol.

The (illustrative) SpO2 data packets are received by the first radio 12 in the magnet room 22, and transmitted to the second radio 14 in the control room 30 via the fiber optic cable 16. However, the carrier frequency F_{SPO2} may contain traffic from other devices (e.g., other sensors 18, the patient monitor 32, the imaging device 24, and the like) located in the magnet room 22. When the door 37 between the magnet room 22 and the control room 30 is open, the sensors 18, the second radio 14, and the patient monitor radio 36 may all be in range of each other. Therefore, it is important that the second radio 14 located in the control room 30 be configured transmit at a specific time so as not to interfere with the other packet traffic. Time synchronization between the two radios can be achieved using an Ethernet protocol, application level packets, or other packet synchronization protocol.

After another idle time period of approximately 1.038 milliseconds, the data stream is transmitted from other equipment (e.g., the other sensors 18, the patiet monitor 32, an infusion pump, a ventilator, an anesthesia machine, and the like) in the magnet room 22. This transmission occurs during a first other channel time period 52, which lasts approximately 224 microseconds. In addition, after another idle time period of approximately 1.776 milliseconds, the second radio 14 can send the data packet directly to the patient monitor radio 36 during a repeater time period 54 which lasts approximately 584 microseconds. In this manner, the second radio 14 is receiving the same data stream from different sources (i.e., the first radio 12 and the sensor 18) at different times to avoid other traffic.

After yet another idle time of approximately 1.416 milliseconds, the other equipment is configured to send the data packets to the patient monitor 32 during a second other channel time period 56, which lasts approximately 224 microseconds. Again, the idle period allows the second radio 14 to transmit the data to the patient monitor 32 to avoid other data transmission traffic. In some examples, when the patient monitor 32 is located in the control room 30, back channel packets can be sent to the magnet room 22 or retransmission to the sensor 18 at the appropriate time. Finally, after another idle period that lasts approximately 1.776 milliseconds, the process repeats with the sensor 18 transmitting another data packet to the first radio 12 in the forward channel during a forward channel time period 48.

In this manner, in the magnet room 22, the patient monitor 32 can directly receive original sensor data from the sensor 18. When the patient monitor 32 is moved to another location, such as the doorway between the magnet room 22 and the control room, the patient monitor 32 can receive both the original and repeated forward channel packets. In the control room 30 with the door 37 closed, the patient monitor 32 can receive repeated packets only. This allows the patient monitor 32 to be moved back and forth between the rooms 22, 30 and continuously display patient information.

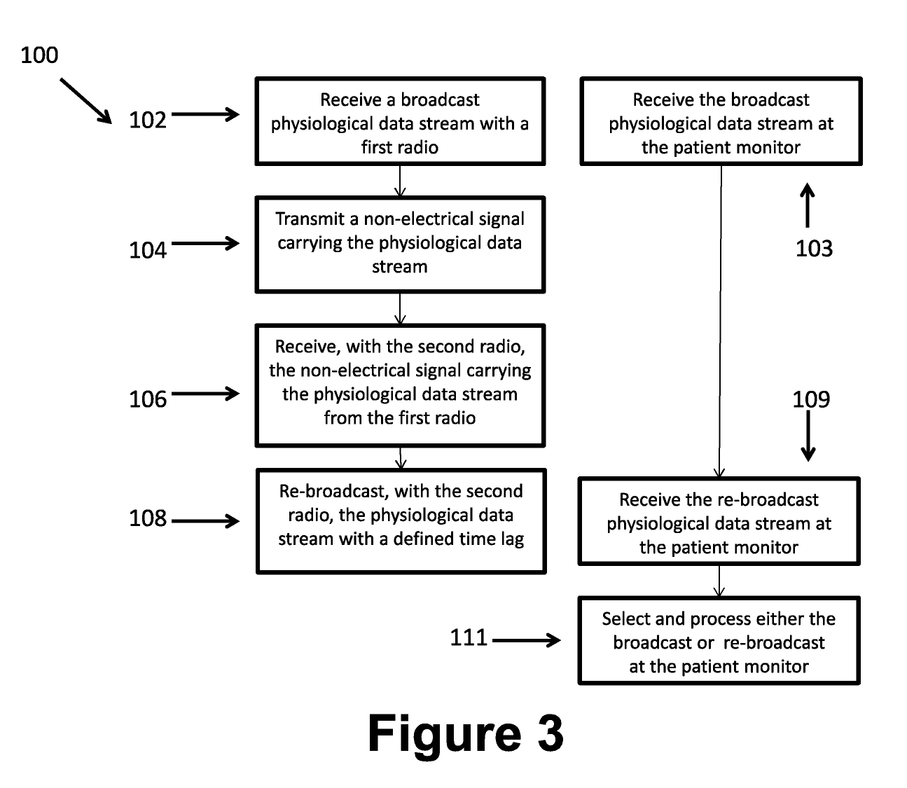

FIG. 3 shows a method 100 of for processing physiological data streams broadcast by at least one associated physiological sensor 18. The method 100 includes receiving, with a first hub 6 including a first radio 12, an individual physiological data stream broadcast by the at least one associated physiological sensor 18 at a data stream carrier frequency (102). At the same time, the broadcast is also received by the patient monitor 32 (103), if the patient monitor is in range (e.g. inside the magnet room 22). With the first radio 12, a non-electrical signal carrying the individual physiological data stream is transmitted to a second hub 8 including a second radio 14 via a non-electrical communication link 16 between the first radio 12 and the second radio 14 (104). At the second radio 14, the non-electrical signal carrying the physiological data stream is received from the first radio 12 (106). The second radio 14 re-broadcasts the physiological data stream at the data stream carrier frequency with a defined time lag respective to the broadcast by the at least one associated physiological sensor 18 (108). The re-broadcast is received by the patient monitor 32 (109), if the patient monitor is in range (e.g. inside the control room 22). The patient monitor then selects and processes (e.g. displays the data trend from) either the broadcast or the re-broadcast (111), depending upon which has the best signal strength or depending on some other selection criterion.

The various components 6, 8, 12, 14, 18, 32 of the apparatus 10 can include at least one microprocessor 28, 34 programmed by firmware or software to perform the disclosed operations. In some embodiments, the microprocessor 28, 34 is integral to the various component 12, 14, 18, 32, so that the data processing is directly performed by the various component 12, 14, 18, 32. In other embodiments the microprocessor 28, 34 is separate from the various component 12, 14, 18, 32. The data processing components 28, 34 of the apparatus 10 may also be implemented as a non-transitory storage medium storing instructions readable and executable by a microprocessor (e.g. as described above) to implement the disclosed operations. The non-transitory storage medium may, for example, comprise a read-only memory (ROM), programmable read-only memory (PROM), flash memory, or other repository of firmware for the various components 12, 14, 18, 32 and data processing components 28, 34. Additionally or alternatively, the non-transitory storage medium may comprise a computer hard drive (suitable for computer-implemented embodiments), an optical disk (e.g. for installation on such a computer), a network server data storage (e.g. RAID array) from which the various component 6, 8, 12, 14, 18, 32, data processing components 28, 34, or a computer can download the apparatus software or firmware via the Internet or another electronic data network, or so forth.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An apparatus for processing medical information data streams broadcast by at least one associated physiological sensor, the apparatus comprising:
a first hub including:
a first radio configured to receive an individual medical information data stream broadcast by the at least one associated sensor at a data stream carrier frequency to transmit a non-electrical signal carrying the individual medical information data stream;
a second hub including a second radio; and
a fiber optic cable between the first hub and the second hub via which the non-electrical signal carrying the individual medical information data stream is communicated from the first radio to the second radio;
the second radio being configured to receive the non electrical signal carrying the medical information data stream from the first radio and re-broadcast the medical information data stream at the data stream carrier frequency with a defined time lag respective to the broadcast by the at least one associated sensor.

2. (canceled)

3. The apparatus of claim 1, further comprising:
a patient monitor including a radio configured to:
receive the individual medical information data stream broadcast by the at least one sensor at the data stream carrier frequency; and
receive a re-broadcast from the second radio of the medical information data stream at the data stream carrier frequency with the defined time lag.

4. The apparatus of claim 3, wherein:
the first hub includes a multiplexer programmed to:
receive a plurality of individual medical information data streams from one or more sensors;
bundle the plurality of individual medical information data streams into a single data stream; and
transmit the non-electrical signal carrying the single data stream via the fiber optic cable to the second radio; and
the second hub includes a de-multiplexer programmed to:
receive the single data stream from the non-electrical communication link;
de-bundle the single data stream into the individual data streams; and
retransmit the individual data streams to the patient monitor.

5. The apparatus of claim 3, wherein the patient monitor is further configured to select one of the broadcast and the re broadcast and to read the medical information data stream from the selected one of the broadcast and the re broadcast.

6. The apparatus of claim 5, wherein the patient monitor is configured to select one of the broadcast and the re broadcast based on a signal strength metric of at least the broadcast.

7. The apparatus of claim 1, wherein the first hub includes at least one first physical port corresponding to each of the associated sensors, each first port being configured to receive medical information data from the corresponding sensor; and
the second hub includes at least one second physical port corresponding to each of the first ports, each second port being configured to receive the medical information data from the corresponding first port via the fiber optic cable.

8. The apparatus of claim 1, wherein the second hub includes a hospital information system communication link configured to transmit data from the second radio to an associated hospital information system.

9. A magnetic resonance imaging (MRI) system comprising:
an MRI imaging device disposed in a magnet room;
a Faraday cage enclosing the magnet room; and
the apparatus of claim 1 for processing medical information data streams broadcast by at least one associated sensor disposed on a patient in the MRI imaging device, wherein the first hub is disposed inside the Faraday cage, the second hub is disposed outside the Faraday cage, and the fiber optic cable passes through the Faraday cage.

10. A method for processing physiological data streams broadcast by at least one associated physiological sensor, the method comprising:
receiving, with a first hub including a first radio, an individual physiological data stream broadcast by the at least one associated physiological sensor at a data stream carrier frequency;
transmitting, with a first radio, a non-electrical signal carrying the individual physiological data stream to a second hub including a second radio via a fiber optic cable between the first radio and the second radio;
receiving, with the second radio, the non electrical signal carrying the physiological data stream from the first radio; and
re-broadcasting, with the second radio, the physiological data stream at the data stream carrier frequency with a defined time lag respective to the broadcast by the at least one associated physiological sensor.

11. (canceled)

12. The method of claim 10, further comprising:
with a patient monitor including a radio:
receiving the individual physiological data stream broadcast by the at least one physiological sensor at the data stream carrier frequency; and
receiving a re-broadcast from the second radio of the physiological data stream at the data stream carrier frequency with the defined time lag.

13. The method of claim 12, further including:
with the first hub,
receiving a plurality of individual physiological data streams from the at least one associated physiological sensor;
bundling each physiological data stream into a single data stream; and
transmitting the non-electrical signal carrying the single data stream via the fiber optic cable to the second radio; and
with the second hub,
receiving the single data stream from the fiber optic cable;
de-bundling the single data stream into the individual data streams; and
retransmitting the individual data streams to the patient monitor.

14. The method of claim 13, further including:
with the patient monitor, selecting one of the broadcast and the re broadcast and to read the physiological data stream from the selected one of the broadcast and the re broadcast.

15. The method of claim 14, further including:
with the patient monitor, selecting one of the broadcast and the re broadcast based on a signal strength metric of at least the broadcast.

16. The method of claim 10, wherein
with at least one first physical port of the first hub corresponding to each of the associated physiological sensors, receiving physiological data from the corresponding physiological sensor; and
with at least one second physical port of the second hub corresponding to each of the first ports, receiving the physiological data from the corresponding first port via the fiber optic cable.

17. The method of claim 10, further including:
with a hospital information system communication link of the second hub, transmitting data from the second radio to an associated hospital information system.

18-20. (canceled)
